(19) United States
(12) Patent Application Publication
(10) Pub. No.: US 2004/0242642 A1
(43) Pub. Date: Dec. 2, 2004

(54) Crystalline esomeprazole compounds and process for the preparation thereof

(75) Inventors: Manne Satyanarayana Reddy, Hyderabad (IN); Muppa Kishore Kumar, Hyderabad (IN); Koilkonda Purandhar, Hyderabad (IN); Lekkala Amarnath Reddy, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/716,200

(22) Filed: Nov. 18, 2003

(30) Foreign Application Priority Data
Nov. 18, 2002 (IN) 852/MAS/2002

(51) Int. Cl. C 07D 4 3/02; A61K 31/4439

(52) U.S. Cl. 514/338; 546/273.7

(57) ABSTRACT

The present invention relates to crystalline form II of esomeprazole trihydrate and methods of its preparation and use. Preferably, the crystalline form II of esomeprazole trihydrate has an X-ray powder diffraction pattern that includes five or more peaks with 2 theta angles of 4.82±0.09, 5.55±0.09, 7.41±0.09, 8.60±09, 12.10±0.09, 14.16±0.09, 18.47±0.09, and 21.08±0.09.

### CROSS REFERENCE TO RELATED APPLICATIONS

This application claims priority of Indian Patent Application No. 852/MAS/2002, filed Nov. 18, 2002, the content of which is incorporated by reference in its entirety.

### FIELD OF THE INVENTION

The present invention relates to a crystalline form of hydrated esomeprazole salt and in particular to esomeprazole magnesium trihydrate salt, chemically known as (S)(-)5-methoxy-2-[[(4-methoxy-3,5-dimethyl-2-pyridinyl)-methyl]sulphinyl]-1H-benzimidazole trihydrate.

### BACKGROUND OF THE INVENTION

Esomeprazole is the (S)(-) enantiomer of omeprazole, a sulfoxide which has an asymmetric center at the sulfur atom and exists as optical isomers (enantiomers). Esomeprazole ((S)(-)5-methoxy-2-[[(4-methoxy-3,5-dimethyl-2-pyridinyl)-methyl]sulphinyl]-1H-benzimidazole) is the S(-)enantiomer of the drug omeprazole. Known forms of esomeprazole, and its salts, hydrates, and polymorphs, are gastric acid secretion inhibitors.

Omeprazole, and its therapeutically acceptable alkaline salts are disclosed in EP 000 5129 and EP 124,495 respectively, while DE 4035455 discloses separation of the enantiomers of omeprazole using diastereomeric ether. WO 00/44744 discloses the potassium salt of esomeprazole. U.S. Pat. No. 6,162,816 discloses crystalline form A and less crystalline form B of neutral esomeprazole, prepared by a recrystallization from ethyl acetate, methylene chloride or toluene.

U.S. Pat. No. 6,369,085, which is incorporated herein by reference in its entirety, discloses esomeprazole magnesium trihydrate prepared from the corresponding potassium salt, precipitated with acetone, and treated with water. The crystalline form of the '085 patent will be designated herein as crystalline form I.

A number of drugs have been found to exhibit desirable dissolution characteristics and, in some cases, desirable bioavailability patterns when used in a specific solid form, e.g., as an amorphous or crystalline solid. Therefore, there is a continuing need for new solid forms of esomeprazole and methods of their preparation.

### SUMMARY OF THE INVENTION

In one aspect, the invention provides a compound which is a crystalline form II of esomeprazole magnesium trihydrate. Preferably, the compound of this aspect of the invention has an X-ray powder diffraction pattern expressed the terms of 2 theta angles and obtained with a diffractometer equipped with a copper K X-radiation source, wherein said X-ray powder diffraction pattern includes five or more peaks selected from the group consisting of peaks with 2 theta angles of 4.82±0.09, 5.55±0.09, 7.41±0.09, 8.60±09, 12.10±0.09, 14.16±0.09, 18.47±0.09, and 21.08±0.09.

In another aspect, the invention provides a composition comprising solid esomeprazole magnesium, wherein at least 75% of said esomeprazole magnesium is a crystalline form II of esomeprazole magnesium tribydrate. Preferably, the composition is substantially free of other forms of esomeprazole and is a solid powder of bulk esomeprazole magnesium for use as an active pharmaceutical ingredient.

In yet another aspect, the invention provides a process for making a trihydrate of esomeprazole magnesium in the form of a crystalline solid that includes:

a) providing esomeprazole magnesium as a solution in a ketone-containing solvent;

b) cooling the solution so that a solid mass separates; and

c) isolating the separated solid mass, which is the trihydrate of esomeprazole magnesium in the form of a crystalline solid. The preferred alcohol-containing solvent is methanol, ethanol, propanol, and butanol; methanol is more preferred. The preferred ketone-containing solvent is a mixture of acetone and water. In a more preferred embodiment of this aspect, the invention provides a process for making a trihydrate of esomeprazole magnesium in the form of a crystalline solid that includes:

a) providing esomeprazole magnesium in methanol;

b) contacting the esomeprazole magnesium in methanol with water so that a solid mass separates;

c) isolating the solid mass by filtration;

d) washing the solid mass;

e) dissolving the solid mass in methanol and filtering the solution so formed to separate excess magnesium solids;

f) removing solvent from the solution to obtain isolated residual mass;

g) re-precipitating the isolated residual mass from a mixture of acetone and water, and

h) drying the isolated residual mass, which is the trihydrate of esomeprazole magnesium in the form of a crystalline solid.

The invention also provides a pharmaceutical composition containing the crystalline form II of esomeprazole magnesium trihydrate, and methods of administration related thereto.

### BRIEF DESCRIPTION OF THE DRAWINGS

FIG. 1 shows an example of x-ray powder diffraction pattern (XRD) for crystalline form II of esomeprazole magnesium trihydrate.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

For purposes of the present invention, the following terms are defined below. “Pharmaceutically acceptable” means that which is useful in preparing a pharmaceutical composition that is generally non-toxic and is not biologically undesirable and includes, but is not limited to, that which is customarily utilized for veterinary use and/or human pharmaceutical use.

The term “composition” includes, but is not limited to, a powder, a solution, a suspension, a gel, an ointment, an emulsion and/or mixtures thereof. The term composition is intended to encompass a product containing the specified ingredient(s) in the specified amount(s), as well as any product, which results, directly or indirectly, from combination of the specified ingredients in the specified amounts. A “composition” may contain a single compound or a mixture of compounds. A “compound” is a chemical substance that includes molecules of the same chemical structure.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the crystalline solid described herein, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

“Therapeutically effective amount” means the amount of a compound that, when administered for treating or preventing a disease, is sufficient to effect such treatment or prevention for the disease. The “therapeutically effective amount” will vary depending on the compound, the disease and its severity and the age, weight, etc., of the patient to be treated.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “substantially free of” in reference to a composition, as used herein, means that the substance from which the composition is free of cannot be detected by methods known to those skilled in the art.

“About” means the exact number modified by the word, and in addition a reasonable range of values surrounding that number, as would be recognized by a skilled person as forming a reasonable range with regard to the number measured, the property measured, synthesis, detectability, operating parameters of instruments, and other relevant factors.

The term “solvent” may be used to refer to a single solvent or a mixture of solvents. An “alcohol-containing solvent” means a solvent which contains an alcohol. For example, a single alcohol, a mixture of different alcohols, and a mixture of an alcohol with one or more non-alcohol solvents, which non-alcoholic solvents may be organic or aqueous, all qualify as “alcohol-containing solvent(s).” The term “non-aqueous solvent” and the term “organic solvent” may be used interchangeably to mean a solvent conventionally understood as such in the art, including a solvent in which non-polar or hydrophobic compounds are preferentially and substantially soluble. The term “aqueous solvent” preferably means a solvent containing water, or a solvent in which polar or hydrophilic compounds are preferentially and substantially soluble. The term “haloalkane” means an alkane with one or more halogen substituents, which alkane may have one to six carbons, preferably one to three carbons, and be branched or unbranched.

In one aspect, the invention provides a new crystalline form of esomeprazole magnesium trihydrate salt herein designated a crystalline form II, which is believed to have a unique X-ray diffractogram. Esomeprazole ((S)(-)5-methoxy-2-[[(4-methoxy-3,5-dimethyl-2-pyridinyl)-methyl]sulphinyl]-1 H-benzimidazole; the S(-)enantiomer of omeprazole), as well as its salts, is an active gastric acid secretion inhibitor. Preferably, the crystalline Form II of esomeprazole magnesium is substantially free of R-omeprazole magnesium salt. It can be synthesized with good reproducibility, favoring large-scale production. It is also obtainable as a free flowing and non-solvated crystalline solid, a form useful in pharmaceutical formulations.

Different solid forms of the same drug may exhibit different properties, including characteristics that have functional implications with respect to their use as active ingredients of pharmaceutical products. For example, polymorphs of the same drug may have substantial differences in such pharmaceutically important properties as dissolution rates and bioavailability. Likewise, different polymorphs may have different processing properties, such as hydroscopicity, flowability, and the like, which could affect their suitability as active pharmaceuticals for commercial production.

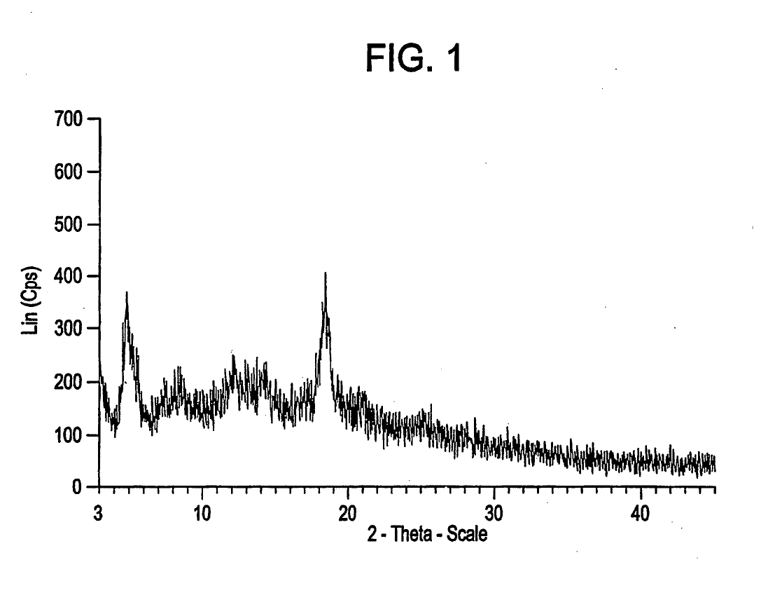

For crystalline compounds, XRD (x-ray powder diffraction) is a useful characterization tool. Each XRD is unique for the particular crystalline form. Each crystalline form exhibits a diffraction pattern with a unique set of diffraction peaks that can be expressed in 2 theta angles, d-spacing values and relative peak intensities. 2 theta diffraction angles and corresponding d-spacing values account for positions of various peaks in the XRD pattern, and d-spacing values may calculated with observed 2 theta angles and copper K(α1) wavelength by well known methods using the Bragg equation. FIG. 1 shows the X-ray diffractogram of one batch of solid crystalline form II of esomeprazole magnesium trihydrate obtained by the inventors (the process of making the solid crystalline form II of esomeprazole magnesium trihydrate is described in greater details below). The x-ray powder diffractogram was measured on a Bruker Axs, D8 Advance X-ray Powder diffractometer with Cu K alpha-1 radiation source. The XRD data for the crystalline form II of esomeprazole magnesium trihydrate as obtained by the inventors are as follows:

| 2-theta value | Relative Intensity (%) |
|---|---|
| 4.824 | 100.0 |
| 18.471 | 81.7 |
| 5.552 | 43 |
| 14.16 | 28.1 |
| 12.104 | 25.3 |
| 8.608 | 22.3 |
| 21.089 | 21.5 |
| 7.411 | 18.8 |

The crystalline form II of esomeprazole magnesium trihydrate may be identified by x-ray diffraction. One method of identifying particular crystalline forms is to compare their XRD patterns. If the comparison shows that the two patterns are similar to each other within a technically reasonable range as a skilled person would understand, then the two compared crystalline forms are substantially the same. For example, one skilled in the art can overlay an XRD pattern of an unidentified crystalline form obtained using the methods described herein, over the XRD in FIG. 1 and readily determine whether the XRD pattern of the unidentified form is substantially the same as the XRD pattern in FIG. 1. Of course, slight variations in observed 2 theta angles or d-spacing values are expected based on the specific diffractometer employed and the sample preparation technique. More variation is expected for the relative peak intensities. Therefore identification of the exact crystal form of a compound should be based primarily on observed 2 theta angles with lesser importance attributed to relative peak intensities. For these reasons, some margin of error is present in each of the 2 theta angle assignments and d-spacings reported herein. The margin of error assigned herein for the 2 theta angles is approximately ±0.09 for each of the peak assignments. For this reason, the crystalline form II of esomeprazole magnesium trihydrate is believed to have an X-ray powder diffraction pattern that includes five or more peaks selected from the group consisting of peaks with 2 theta angles of 4.82±0.09, 5.55±0.09, 7.41±0.09, 8.60±09, 12.10±0.09, 14.16±0.09, 18.47±0.09, and 21.08±0.09.

Although 2 theta angles or d-spacing values are the primary methods of identifying the crystalline form, it may be desirable to also compare relative peak intensities. As noted above, relative peak intensities may vary depending upon the specific diffractometer employed and the sample preparation technique. The peak intensities are reported as intensities relative to the peak intensity of the strongest peak. The margin of error for the relative intensities reported herein is approximately 10% to 15%.

In another aspect, the invention provides a composition including esomeprazole magnesium as a solid, in which at least 80%, preferably 90%, more preferably 95%, and most preferably 99% by weight of the crystalline form II of esomeprazole magnesium trihydrate. The remainder of the esomeprazole magnesium in the composition, e.g., 20%, preferably 10%, more preferably 5%, and most preferably 1% or less of the total weight of esomeprazole magnesium, may be amorphous or one or more other crystalline forms of esomeprazole magnesium. In one embodiment of this composition, the solid esomeprazole magnesium is substantially free from amorphous forms of esomeprazole magnesium. In yet another embodiment, in addition to the crystalline form II of esomeprazole magnesium trihydrate, the composition includes at least a small amount of an amorphous form of esomeprazole magnesium. In a non-limiting example, the composition includes 95% of the crystalline form II of esomeprazole magnesium trihydrate and at least 1% of an amorphous form of esomeprazole magnesium. In another non-limiting example, the composition includes at least 80% of the trihydrate of esomeprazole magnesium in the form of an crystalline solid and at least 5% of the amorphous form of esomeprazole magnesium. All compositions, in 0.1% increments, which include at least 80% of the crystalline form II of esomeprazole magnesium trihydrate and at least 1% of the amorphous form of esomeprazole magnesium, are contemplated. All percentages are based upon the total amount of the solid esomeprazole magnesium in the composition. The preferred form of the composition of this aspect of the invention is a solid powder of bulk esomeprazole magnesium for use as an active pharmaceutical ingredient. This powder composition has a moisture content, which is preferably from about 7% to about 8%. Moisture content may be measured by any accepted technology, for example by using Karl Fischer reagent (KF) and an appropriate instrument (goniometer) such as a Mettler DL-35, a Scintag PAD V, a Brukker D5000, or by thermogravimetric analysis using moisture analysis instruments such as the Mettler DSC20, TG50, and TC10A.

To determine the relative amounts of amorphous and crystalline components in the composition of this aspect of the invention, one suitable analytical methodology is X-ray powder diffraction (XRD). XRD methodology is capable of providing both qualitative and quantitative information about compounds present in a solid sample. XRD is adaptable to quantitative applications because the intensities of the diffraction peaks of a given compound in a mixture are proportional to the fraction of the material in the mixture. By measuring the intensity of the diffraction lines and comparing them with standards, it is possible to make a quantitative analysis of crystalline mixtures.

As explained above, amorphous solids have no characteristic peaks. In contrast, each crystalline solid is arranged in a set of planes separated by interplanar space d, and exhibits a diffraction pattern with a unique set of peaks generated when x-rays strike a plane at angle theta and are diffracted at the same angle, thus the 2 theta angle is determined by the spacing between a particular set of planes. The identification of a crystalline solid is based upon peaks in the XRD pattern being tabulated in terms the diffraction angle 2 theta (or d-spacing) and their relative intensities. Identification of a crystal form of a compound should be based primarily on observed 2 theta angles with lesser importance being attributed to relative peak intensities. Different quantitative techniques are available. For example, two methods may be used to analyze XRD quantitatively: the Internal Standard Method and the External Standard Method. The Internal Standard Method is the preferred procedure for analyzing powdered systems. This method measures a known quantity of a reference powder which is added to an unknown powder. The mass absorption coefficient of the mixture need not be known in advance. Any number of constituents in the mixture may be quantified independently, including the amorphous (non-crystalline) components. The External Standard Method is used to analyze solid systems when the mass absorption co-efficient is known. It allows the quantification of one or more components in a system, which may contain an amorphous fraction. The percent composition of a crystalline compound can be determined in an unknown composition. The XRD patterns of an unknown composition can be compared to a known standard containing pure crystalline compound to identify the percent ratio of the crystalline form of the compound. This is done by comparing the relative intensities of the peaks from the diffraction pattern of the unknown composition with a calibration curve based on the XRD pattern for the strongest peak derived from the XRD pattern of a pure crystalline sample of the compound. The peak intensities are reported as intensities relative to the peak intensity of the strongest peak (“the 100% peak”). The calibration curve may be created in a manner known to those of skill in the art. For example, five or more artificial mixtures of amorphous and crystalline forms of crystalline compound in different amounts, may be prepared. As an example, such mixtures may contain, 2%, 5%, 7%, 8%, and 10% of crystalline compound, with the remainder being the amorphous form of the salt. Then, XRD patterns are obtained for each artificial mixture using standard XRD techniques. Slight variations in peak positions, if any, may be accounted for by adjusting the location of the peak to be measured. The intensities of the 100% peak(s) for each of the artificial mixtures are then plotted against the known weight percentages of the crystalline form. The resulting plot is a calibration curve that allows determination of the amount of crystalline compound in an unknown sample. For the unknown mixture of crystalline and amorphous compounds, the intensities of the 100% peak(s) in the mixture, relative to an intensity of this peak in a calibration mixture, may be used to determine the percentage of the crystalline form in the composition, with the remainder determined to be the amorphous material.

In order to determine the relative amount of amorphous to crystalline solid in the compositions of this invention, XRD information may be used to create the calibration curve(s) described above. For use in this comparative analysis, XRD patterns of crystalline forms of esomeprazole are obtainable by known methods of measurement. For example, the XRD data for crystalline Form I esomeprazole magnesium trihydrate is disclosed in U.S. Pat. No. 6,369,085, which is incorporated by reference for this purpose.

In another aspect, the invention provides a process for making the crystalline form II esomeprazole magnesium trihydrate by a) providing esomeprazole magnesium as a solution in a ketone-containing solvent; b) cooling the solution so that a solid mass separates; and c) isolating the separated solid mass, which is the trihydrate of esomeprazole magnesium in the form of a crystalline solid; particularly, the crystalline form II of esomeprazole magnesium trihydrate. The starting materials and reagents used in this process are commercially available and/or may be readily synthesized by a skilled person, unless otherwise indicated. Esomeprazole base may be made as known in the art. See in addition U.S. Pat. Nos. 6,162,816 and 5,693,818, which are incorporated herein by reference. Any conventional aqueous or organic solvent that would not hinder or would contribute to the reactions by which the process of the invention proceeds may be included in the ketone-containing solvent. Non-limiting examples of suitable ketones include acetone, ethyl methyl ketone, methyl isobutyl ketone, and diethyl ketone. The preferred ketone-containing solvent is a mixture of acetone and water. Examples of organic solvents include chlorinated alkanes, such as chloroform, dichloromethane, dichloroethane, and carbon tetrachloride; ester solvents such as lower alkyl esters of organic acids, such as methyl, ethyl, propyl isopropyl, butyl, isobutyl, and tert-butyl acetate; and nitriles, such as acetonitrile. Preferably, the alcohol component of the alcohol-containing solvent is preferably methanol, ethanol, propanol, or butanol, more preferably ethanol, n-propanol, tert-butanol, n-butanol, and most preferably methanol. The alcohol-containing solvent may be a pure alcohol (for example, methanol) or may be a mixture of alcohol with other solvent(s), for example with water, with a ketone solvent such as acetone, or with both. Preferably the alcohol-containing solvent includes methanol.

Certain operational steps are well known in the art and, unless otherwise indicated, any known method for performing these functions may be used in the processes of this invention. For example, solvents may be removed by distillation in atmosphere or under vacuum. Drying may be accomplished by evaporation, spray drying, drying under vacuum, and freeze-drying. Stirring means any method for blending or mixing a reaction mixture. Reagents and/or reaction mixtures may be combined by adding one to the other, for example, water may be poured into a reaction mixture. Esomeprazole magnesium may be provided, for example, by suspending magnesium metal in an alcohol-containing solvent in the presence of a haloalkane and adding esomeprazole base (which may itself be dissolved in an alcohol-containing solvent). Preferred haloalkanes are dichloromethane, dichloromethane (in particular 1,2-dichloroethane) and trichloromethane (chloroform); most preferably, dichloromethane. The process then continues by contacting with water. Contacting with water may be accomplished by pouring water into the esomeprazole magnesium solution, or by pouring the esomeprazole magnesium solution into water, or by other conventional methods. The preferred amounts of alcohol-containing solvent and of water in milliliters (ml) may be determined relative to the amount of the starting esomeprazole magnesium (i.e., the esomeprazole magnesium in the alcohol-containing solvent provided in the first step of the process) in grams (g). The amount of alcohol-containing solvent is preferably about 5 ml to about 10 ml per 1 gram of the starting esomeprazole magnesium, preferably about 6 to about 7 ml. The amount of water is preferably about 5 ml to about 25 ml per 1 gram of the starting esomeprazole magnesium, preferably, about 18 ml.

The resulting solid mass of amorphous esomeprazole magnesium may be then recrystallized from the ketone-containing solvent (e.g., acetone/water mixture) to obtain the crystalline form II esomeprazole magnesium trihydrate. The solid mass is isolated, washed by a suitable solvent, such as water or a ketone solvent such as acetone, preferably once with water and once with acetone. However the solid mass may be washed sequentially in these solvents in any combination, for example twice with water and once with the ketone solvent, or the reverse, and so on. It is also helpful to dissolve the isolated solid mass (preferably after filtration, or after filtration and washing) in an alcohol such as methanol. At this stage the solution formed by dissolving the solid mass in the alcohol may be filtered to separate the excess magnesium, which may then be removed by conventional methods. The solution formed by dissolving the isolated solid mass in alcohol is treated to obtain solid material again in the form of an isolated mass. Solvent may be removed from the solution to accomplish this, using conventional methods. The isolated residual mass is preferably re-precipitated, for example from an ester solvent such as ethyl acetate.

The specific non-limiting example of this process includes a) disoolving magnesium metal in the alcohol-containing solvent; b) cooling the mass to 5-10 degrees C.; c) adding esomeprazole base as a solution an alcohol-containing solvent (same or different); d) slowly decomposing the reaction mixture by adding water and storring until a mass separates; e) filtering the isolated solid; f) suspending the wet solid in acetone and storring for 1-2 hours at 5-10 degrees C.; g) filtering the solid; h) dissolving the solid (or external esomeprazole amorphous) in an alcohol-containing solvent (same or different); k) removing the solvent; 1) dissolving the residue in a ketone-containing solvent; m) cooling the mass to from −10 to +10 degrees C. until a mass separates; n) filtering the solid, which is the crystalline form II of esomeprazole magnesium trihydrate; and o0 drying the obtained crystalline form II of esomeprazole magnesium trihydrate at 50-100 degrees C., preferably at 50-70 degrees C.

In another aspect, the invention provides pharmaceutical compositions which include the crystalline form II esomeprazole magnesium trihydrate. Pharmaceutical compositions generally contain, in addition to the active compound or compounds, one or more carriers (also called excipients) which ordinarily lack pharmaceutical activity per se, but have various useful properties which can, for example, enhance the stability, sterility, bioavailability, and ease of formulation of a pharmaceutical composition. These carriers are pharmaceutically acceptable, meaning that they are not harmful to humans or animals when taken appropriately and are compatible with the other ingredients in a given formulation. The carrier may be solid, semi-solid, or liquid, and may be formulated with the compound in bulk, but ultimately in the form of a unit-dose formulation (i.e., a physically discrete unit containing a specific amount of active ingredient) such as a tablet or capsule.

The pharmaceutical compositions of this invention are contemplated in various formulations suitable for various modes of administration, including but not limited to inhalation, oral, rectal, parenteral (including subcutaneous, intradermal, intramuscular, intravenous), implantable, intravaginal and transdermal administration. The most suitable route of administration in any given case depends on the duration of the subject's condition, the length of treatment desired, the nature and severity of the condition being treated, and the particular formulation that is being used. The formulations may be in bulk or in unit dosage form, and may be prepared by methods well known in the art for a given formulation.

The amount of active ingredient included in a unit dosage form depends on the type of formulation in which the active ingredient is presented. A pharmaceutical composition will generally contain about 0.1% by weight to about 99% by weight of the crystalline form II of esomeprazole magnesium trihydrate, preferably about 1% by weight to 50% by weight for oral administration and about 0.2% by weight to about 20% by weight for parenteral administration.

Formulations suitable for oral administration include capsules (hard and soft), cachets, lozenges, syrups, suppositories, and tablets, each containing a pre-determined amount of the active compound; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the active compound and a suitable carrier or carriers. The amount of active ingredient per unit dosage of solid formulations is preferably from about 5 mg to 60 mg, in particular about 8 to 10 mg, about 16 to 20 mg, and about 32 to 40 mg. For liquid oral formulations, a preferable amount is from about 2% by weight to about 20% by weight. Suitable carriers include but are not limited to fillers, binders, lubricants, inert diluents, surface active/dispersing agents, flavorants, antioxidants, bulking and granulating agents, adsorbants, preservatives, emulsifiers, suspending and wetting agents, glidants, disintegrants, buffers and pH-adjusting agents, and colorants. Examples of carriers include celluloses, modified celluloses, cyclodextrins, starches, oils, polyols, sugar alcohols and sugars, and others. For liquid formulations sugar, sugar alcohols, ethanol, water, glycerol, and poyalkylene glycols are particularly suitable, and may also be used in solid formulations. Cyclodextrins may be particularly useful for increasing bioavailability. Formulations for oral administration may optionally include enteric coatings known in the art to prevent degradation of the formulation in the stomach and provide release of the drug in the small intestine. Examples of suitable controlled release formulation vehicles are disclosed in U.S. Pat. Nos. 3,845,770; 3,916,899; 3,536,809; 3,598,123; and 4,008,719, the disclosures of which are hereby incorporated by reference in their entirety.

Formulations suitable for buccal or sub-lingual administration include lozenges comprising the active compound in a flavored base, usually sucrose and acacia or tragacanth, although other agents are also suitable, and pastilles comprising the compound in an inert base such as gelatin and glycerin or sucrose and acacia.

Formulations of the present invention suitable for parenteral administration comprise sterile aqueous and non-aqueous injection solutions of the active compound, preferably isotonic with the blood of the intended recipient. The amount of active ingredient is preferably a concentration of from about 0.1% by weight to 10% by weight. These preparations may contain, among other ingredients, anti-oxidants, buffers, bacteriostats, and solutes that render the formulation isotonic with the blood of the intended recipient. Aqueous and non-aqueous sterile suspensions may include, among others, suspending and thickening agents. The formulations may be presented in unit-dose or multi-dose containers, e.g. sealed capsules and vials, and may be stored in a freeze-dried or lyophilized condition requiring only the addition of the sterile liquid carrier, for example, saline or water-for-injection immediately prior to use. Extemporaneous injection solutions and suspensions may be prepared from sterile powders, granules and tablets of the kind previously described.

Formulations suitable for rectal administration are preferably presented as unit dose suppositories. These may be prepared by admixing the active compound with one or more conventional solid carriers, e.g. cocoa butter, and then shaping the resulting mixture.

Formulations suitable for transdermal delivery include ointments, creams, lotions, and oils and contain well known pharmaceutically and cosmetically suitable ingredients. Bases for such formulations include for example alcohols, lanolin, petrolatum, paraffin, polyethylene glycol, emulsifiers, penetration enhancing agents, and oleaginous vehicles such as oils. Skin patches may also be used, typically consisting of a fabric or paper base impregnated with a suitable dose in a transdermal formulation. Formulations suitable for transdermal administration may also be delivered by iontophoresis, and typically take the form of an optionally buffered aqueous solution of the active compound.

Also part of this invention are methods of treatment using one or more of the compounds of this invention and the pharmaceutical compositions of this invention. In particular, the crystalline form II of esomeprazole magnesium trihydrate may be administered to a subject in an amount effective to reduce secretion of gastric acid by that subject. Although it is possible to use compounds and compositions of this invention to prevent secretion of gastric acid by establishing a dosage level effective to do so, such treatment would only be applicable in special cases, since to alleviate or eliminate most of the conditions discussed above which are treated with the compounds of this invention, gastric acid secretion should not be eliminated altogether, but only reduced in amount or duration. In general, the treatment may be determined to alleviate, to eliminate, or to prevent a given condition based on factors determinable by a skilled physician as discussed below in the context of determining an effective amount for dosage. Further, the compounds of this invention may be administered to a subject for treating a disorder caused by gastric acid secretion by administering to a subject an amount effective to reduce gastric acid secretion by said subject.

The compounds and compositions of this invention may be used for treatment of any specific disorder or condition related to other conditions known to be suitable for treatment by omeprazole compounds. These compounds and compositions are useful for ameliorating or preventing conditions related to secretion of gastric acid, such as ulcers (including those caused by H. pylori), heartburn, gastro-esophageal reflux, esophagitis, hypersecretory conditions (e.g. Zollinger-Ellison, endocrine adenoma, systemic mastocytosis), gastritis, duodenitis, dyspepsia, acute gastrointestinal bleeding (especially upper), for patients on NSAID therapy or in intensive care, to reduce or prevent gastric acid aspiration and stress ulceration. These compounds are also useful for treating inflammatory conditions such as psoriasis and lysosomal enzyme problems, and infections such as those caused by H. pylori.

By subject is meant a human or an animal, preferably human. Animals contemplated by this invention include any animal safely treatable by compounds of this invention, preferably mammals such as bovines, ovines, caprines, equines, felines, canines, rodents, leporids, and other mammalian farm and zoo animals or domestic pets. The effective amount (i.e. dosage) of active compound for treatment will vary depending on the route of administration, the condition being treated, its severity, and duration, and the state and age of the subject. A skilled physician will monitor the progress of the subject and will adjust the dosage accordingly, depending on whether the goal is to eliminate, alleviate, or prevent a given condition. Generally, the starting dosage may be low, but must at least start from the low end of the effective range, and in cases of severe ulcers it may be increased, and the active substance may be administered as maintenance therapy. The dosage of the active compound may be towards the high end of the effective range, or if needed even higher, but should be considered in proportion to the subject's weight. Depending on the solubility of the particular formulation of active compound administered, the daily dose may be divided among one or several unit dose administrations. Administration of the active compounds may be carried out therapeutically, i.e. as a rescue treatment, or prophylactically, and may be maintained for prolonged periods of time. One skilled in the art will take such factors into account when determining dosage. In general oral and parenteral dosages will be in the range of about 5 to about 350 to 400 mg per day of active ingredient, preferably about 8 mg to about 60 mg, most preferably about 10 mg to about 40 mg.

Unless stated to the contrary, words and phrases such as “including,” “containing,” “comprising,” “having”, “for example”, “i.e.”, “in particular” and the like, mean “including without limitation” and shall not be construed to limit any general statement that it follows to the specific or similar items or matters immediately following it. Any values presented as exemplary values are intended to be used for purposes of illustration. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

The Examples provided below are illustrative and are not intended to limit the scope of the claimed invention.

### EXAMPLE 1

### Preparation of Novel Crystalline Form II of Esomeprazole Magnesium Trihydrate Salt from Crude Esomeprazole

Magnesium metal (2.08 grams) was suspended in a mixture of methanol (150.00 ml) and dichloromethane (5.0 ml) and cooled to a temperature of 5-10° C. Then, to the resulting reaction mixture crude esomeprazole (50.0 grams)] dissolved in methanol (150.0 ml) was added. The resulting reaction mixture was slowly decomposed by adding water (900 ml) and stirred until a solid results. The resulting solid mass was filtered and washed with water (300 ml). The wet solid was suspended in acetone (200 ml) and stirred at a temperature of 0-5° C. until the solid separated. The separated solid was filtered and washed with acetone (50 ml). Further the wet solid was dissolved in methanol (300 ml) and filtered. Then by expelling the resulting filtrate white solid was isolated. The isolated white solid was recrystallised from a mixture of water (175 ml) and acetone (175 ml). The crystallized solid was dried at a temperature of 60-65° C. to afford the title compound. [Weight: 8.5 grams).

### EXAMPLE 2

### Preparation of Crystalline Form II of Esomeprazole Magnesium Trihydrate Salt from Amorphous Form of Esomeprazole

The amorphous form of esomeprazole magnesium salt (25 grams) was dissolved in methanol (100.00 ml). The resulting reaction solution was filtered through a high-flow bed and the bed washed with methanol (50 ml). Then the solvent was distilled off completely from the reaction solution, water (50 ml) added to the resulting residue and the reaction mixture stirred until the solid resulted. The resulting solid mass was suspended in mixture of water (300 ml) and acetone (300 ml), and stirred at a temperature of 0-5° C. until solid separated. THe separated solid was filtered and washed with mixture of water (50 ml) and acetone (50 ml). Further the wet solid was suck dried at a temperature of 60-70° C. to afford the title compound. [Weight: 14.8 grams). The exemplified compounds have a moisture content in the range of 7.0 to 8.0% measured on a Mettler DL-35 using Karl-Fischer reagent by the Karl Fischer method. This moisture content indicates a trihydrate salt.

Unless stated to the contrary, words and phrases such as “including,” “containing,” “comprising,” “having”, “for example”, “i.e.”, “in particular” and the like, means “including without limitation” and shall not be construed to limit any general statement that it follows to the specific or similar items or matters immediately following it. Except where the context indicates to the contrary, all exemplary values are intended to be used for purposes of illustration. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

1. A compound which is a crystalline form II of esomeprazole magnesium trihydrate.

2. The compound of claim 1, having substantially the same X-ray diffraction pattern as shown in FIG. 1.

3. The compound of claim 1, having an X-ray diffraction pattern expressed in terms of 2 theta angles and obtained with a diffractometer equipped with a copper K X-radiation source, wherein said X-ray powder diffraction pattern includes five or more peaks selected from the group consisting of peaks with 2 theta angles of about 4.824, about 5.552, about 7.411, about 8.608, about 12.104, about 14.16, about 18.471, and about 21.089.

4. The compound of claim 1, having an X-ray powder diffraction pattern expressed the terms of 2 theta angles and obtained with a diffractometer equipped with a copper K X-radiation source, wherein said X-ray powder diffraction pattern includes five or more peaks selected from the group consisting of peaks with 2 theta angles of 4.82±0.09, 5.55±0.09, 7.41±0.09, 8.60±09, 12.10±0.09, 14.16±0.09, 18.47±0.09, and 21.08±0.09.

5. The compound of claim 4, wherein the X-ray powder diffraction pattern includes peaks with 2 theta angles of about 4.82, about 5.55, about 7.41, about 8.60, about 12.10, about 14.16, about 18.47, and about 21.09.

7. A composition comprising esomeprazole magnesium, wherein at least 75% of said esomeprazole magnesium is a crystalline form II of esomeprazole magnesium trihydrate.

8. The composition of claim 7, which comprises at least 90% of said esomeprazole magnesium is the crystalline form II of esomeprazole magnesium.

9. The composition of claim 8, wherein at least 95% of said esomeprazole magnesium is the crystalline form II of esomeprazole magnesium.

10. The composition of claim 7, which is substantially free of other forms of esomeprazole magnesium.

11. The composition of claim 7, which is a solid powder of bulk esomeprazole magnesium for use as an active pharmaceutical ingredient.

12. The composition of claim 7, which has a moisture content of from about 2% to about 10% as measured by the Karl Fischer method.

13. The composition of claim 12, which has a moisture content of about 7% to about 8% as measured by the Karl Fischer method.

14. The composition of claim 7, wherein 20% or less by weight of the solid esomeprazole magnesium is in amorphous form.

15. The composition of claim 14, wherein 10% or less by weight of the solid esomeprazole magnesium is in amorphous form.

16. The composition of claim 14, wherein 5% or less by weight of the solid esomeprazole magnesium is in amorphous form.

17. The composition of claim 14, wherein 1% or less by weight of the solid esomeprazole magnesium is in amorphous form.

18. The composition of claim 14, wherein said solid esomeprazole magnesium is substantially free of the amorphous form of esomeprazole magnesium.

19. A process for making a trihydrate of esomeprazole magnesium in the form of a crystalline solid, said process comprising:
a) providing esomeprazole magnesium as a solution in a ketone-containing solvent;
b) cooling said solution so that a solid mass separates; and
c) isolating said separated solid mass, which is the trihydrate of esomeprazole magnesium in the form of a crystalline solid.

20. The process of claim 19, wherein said solution is provided by dissolving amorphous esomeprazole magnesium in said ketone-containing solvent.

21. The process of claim 20, wherein said amorphous esomeprazole magnesium is obtained by suspending magnesium metal in said alcohol-containing solvent in the presence of a haloalkane and adding esomeprazole base thereto.

22. The process of claim 21, wherein said alcohol-containing solvent is a mixture of alcohol and water.

23. The process of claim 21, wherein the alcohol-containing solvent includes an alcohol selected from the group consisting of methanol, ethanol, propanol, and butanol.

24. The process of claim 21, wherein the alcohol-containing solvent includes methanol.

25. The process of claim 21, wherein the haloalkane is selected from the group consisting of dichloromethane, trichloromethane, and dichloroethane.

26. The process of claim 21, wherein the haloalkane is dichloromethane.

27. The process of claim 19, wherein said ketone-containing solvent is a mixture of acetone and water.

28. The process of claim 27, wherein the amount of alcohol-containing solvent is about 5 ml to about 10 ml per 1 gram of the starting esomeprazole magnesium.

29. The process of claim 27, wherein the amount of water is about 5 ml to about 25 ml per 1 gram of the starting esomeprazole magnesium.

30. The process of claim 19, wherein the solid mass is isolated by filtration.

31. A process for making a trihydrate of esomeprazole magnesium in the form of a crystalline solid, said process comprising:
a) providing esomeprazole magnesium in methanol;
b) contacting said esomeprazole magnesium in methanol with water so that a solid mass separates;
c) isolating said solid mass by filtration;
d) washing said solid mass;
e) dissolving said solid mass in methanol and filtering the solution so formed to separate excess magnesium solids;
f) removing solvent from the solution to obtain isolated residual mass;
g) re-precipitating said isolated residual mass from a mixture of acetone and water, and
h) drying said isolated residual mass, which is the trihydrate of esomeprazole magnesium in the form of a crystalline solid.

32. The process of claim 31, wherein the esomeprazole magnesium is provided by suspending magnesium metal in methanol in the presence of dichloromethane and adding esomeprazole base.

33. A compound made by the process of claim 19.

34. A pharmaceutical composition comprising a crystalline form II of esomeprazole magnesium trihydrate and a pharmaceutically acceptable carrier.

35. A method for reducing gastric acid secretion in a subject which comprises administering to the subject an amount of a crystalline form II of esomeprazole magnesium trihydrate effective to reduce gastric acid secretion by said subject.

36. A method for reducing gastric acid secretion in a subject which comprises administering to the subject an amount of a crystalline form II of esomeprazole magnesium trihydrate effective to reduce gastric acid secretion by said subject.
